(19) United States
(12) Patent Application Publication
(10) Pub. No.: US 2014/0088554 A1
(43) Pub. Date: Mar. 27, 2014

(54) DRUG-DELIVERY PUMP WITH INTELLIGENT CONTROL

(71) Applicants: Po-Ying Li, Monrovia, CA (US); Shengtao Li, La Puente, CA (US); Jonathan K. Lee, Montebello, CA (US); Patrick Ryan, Los Angeles, CA (US); Alice Lai, Pasadena, CA (US); Sean Caffey, Hawthorne, CA (US); Mark S. Humayun, Glendale, CA (US)

(72) Inventors: Po-Ying Li, Monrovia, CA (US); Shengtao Li, La Puente, CA (US); Jonathan K. Lee, Montebello, CA (US); Patrick Ryan, Los Angeles, CA (US); Alice Lai, Pasadena, CA (US); Sean Caffey, Hawthorne, CA (US); Mark S. Humayun, Glendale, CA (US)

(21) Appl. No.: 14/035,552

(22) Filed: Sep. 24, 2013

Related U.S. Application Data
(60) Provisional application No. 61/704,946, filed on Sep. 24, 2012.

(51) Int. Cl. A61M 5/172

(52) U.S. Cl. CPC A61M 5/172
USPC 604/506; 604/67

(57) ABSTRACT

Embodiments of the present invention utilize a closed-loop feedback control system to ensure accurate drug delivery. This control system may, for example, utilize a flow sensor to measure the volume of delivery and an intelligent control algorithm to anticipate and compensate for overdoses and underdoses. Feedback control systems in accordance herewith can be applied to any piston- or plunger-driven pump system utilizing sensors that measure flow directly or indirectly. Depending on the mode of operation, fluid may be dispensed continuously or in discrete pulses, and the manner of adjustment or calibration may be tailored to the operational mode in use.

### CROSS-REFERENCE TO RELATED APPLICATIONS

This application claims priority to, and the benefits of, U.S. Ser. No. 61/704,946, filed on Sep. 24, 2012, the entire disclosure of which is hereby incorporated by reference.

### TECHNICAL FIELD

The present invention relates to drug-delivery devices, and in particular to control of piston- or plunger-driven drug-pump devices for accurate dosing.

### BACKGROUND

Subcutaneous drug delivery is employed for treatment of conditions such as diabetes, and typically involves modalities such as syringe injections, pre-filled pen injectors and patient-filled portable insulin pumps. Pre-filled pen injectors provide accurate manual insulin dosing using, for example, a pre-filled, bubble-free glass cartridge. Since the glass cartridges are bubble-free, the priming process is simple for the patient. Since the injection is performed manually, however, patient compliance is a challenge; the patient may not observe proper injection timing and/or fail to follow the dosing prescription. Portable insulin pumps can provide fully controlled insulin delivery, improving patient compliance, and reduced numbers of injections (once every 3 days, for example) and programmable dosing schedules enhance the patient's quality of life. Patch pumps with low pump profiles can be attached to the patient's skin without interfering with daily activities such as including showering, sleeping, and exercising. Because these pumps are typically filled by patients, however, risks arise during the priming procedure. Improperly primed reservoirs may contain large air bubbles and cause the pump to inject too much air into the subcutaneous tissue, which poses serious safety concerns.

Accordingly, portable pumps with small footprints and pre-filled drug reservoirs can address various problems including those discussed above. One of the challenges for pumps utilizing glass vials as drug reservoirs is to provide controlled and accurate drug delivery. This challenge arises due to varying stiction/friction forces between the surface of the plunger and glass vial. Even under the same driving pressure, these variable forces may cause the drug to be delivered at different flow rates for basal (continuous) delivery. It will also make bolus delivery (i.e., delivery of a discrete dose over a short time period) unpredictable from one bolus to the next.

A related problem observed in connection with piston-driven pumps is a characteristic residual “tailing” of the flow rate—that is, if the amount of fluid expelled is plotted as a function of time, the plot will contain an asymmetric peak having a steepened front portion and an extended tail portion. This is evident, for example, in many drug-delivery devices that contain a drug reservoir formed of compliant materials. This tailing effect leads to a longer delivery time and, once again, inaccuracies in delivery volume.

### SUMMARY

Embodiments of the present invention utilize a closed-loop feedback control system to ensure accurate drug delivery. This control system may, for example, utilize a flow sensor to measure the volume of delivery and an intelligent control algorithm to anticipate and compensate for overdoses and underdoses. Feedback control systems in accordance herewith can be applied to any piston- or plunger-driven pump system (hereafter, collectively, “driven” pumps) utilizing sensors that measure flow directly or indirectly. An advantage to this approach is adaptation of the control algorithm to the pump's output, ultimately resulting in extremely accurate drug delivery.

In general overview, a driven pump device in accordance herewith typically includes a cylindrical vial or cartridge with an outlet, and a piston or plunger movable therein. The piston/plunger divides the interior of the vial into a front chamber that is filled with liquid drug and, thus, forms the drug reservoir, and a back chamber that contains the pump mechanism that drives the piston. For example, in electrolytic drug pump devices, the back chamber, or “pump chamber,” may contain a pair of electrodes and an electrolyte from which, upon application of a drive current to the electrodes, electrolysis gas evolves, building up pressure in the chamber that pushes the piston forward so as to expel drug through the outlet. Other pump mechanisms (e.g., osmotic, electrochemical, motor-driven, etc.) may also be used.

In general, the drug flow rate of a piston pump device can be regulated via the drive force/pressure applied by the pump; for electrolytically driven pump devices, for example, this is, in turn, a function of the drive current. The pump can be operated continuously to dispense drug at a desired steady flow rate, or in a “pulsed” manner (i.e., turning the pump on and off at certain intervals for specific periods of time) to deliver a series of discrete drug volumes. (Which mode of operation is used often depends on the drug regimen. For instance, diabetes patients usually need a continuous, low “basal” rate of insulin, in addition to high-rate, short-duration “bolus” deliveries before or after meals.) Sometimes, frequent small-volume bolus injections are used to provide, on average, a very low basal rate; this is called “discrete basal delivery.” As explained above, the actual mechanics of piston pump devices can undermine the accuracy of drug delivery. One problem is the variable stiction/friction between the piston and glass vial, which can cause unstable flow rates despite constant drive pressure. Another problem is the compression of the piston (which is usually made of a rubber-like material) during pump operation, which results, after the pump has been turned off, in a residual “tail” of drug flow as the piston relaxes from its compressed state. This tail can be strongly affected, in addition, by fluid viscosity, which can vary with the particular drug composition as well as the temperature at administration. As a result of this tail, the actual drug volume delivered is larger than the “set” volume, which is the set flow rate during pump operation multiplied by the time period of operation

Embodiments of the present invention address these inaccuracies by measuring the flow rate of drug (with any suitable flow-rate sensor disposed at the drug reservoir outlet or in a cannula, needle, or other fluid conduct downstream thereof) and adjusting pump operation based thereon in real time. For bolus deliveries, the “tail volume” (i.e., the volume of liquid delivered during the residual tail described above) may be measured during a priming stage (before drug is injected into the patient), and the “set volume” decreased such that the sum of tail volume and set volume equals the desired dosage. Similarly, for discrete basal delivery, the set volume for each pulse is adjusted based on the average tail volume of a number of immediately preceding pulses. For “continuous basal delivery” (or simply “continuous delivery”), in which fluid is dispensed continuously rather than in discrete pulses, the accumulated delivered volume may be repeatedly measured for a time window, and deviations of the measured volume from the target volume (i.e., the target flow rate times the length of the time window) are compensated for by adjusting the set flow rate for the next time window (typically between upper and lower flow-rate boundaries).

In a first aspect, the invention pertains to a drug pump device. In various embodiments, the device comprises a drug reservoir; an exit member for fluidically connecting the reservoir with a drug injection site; a flow sensor; an electrolysis pump operable to continuously force fluid from the drug reservoir into the exit member during a continuous pumping cycle; and control circuitry configured to (a) store a range of an operating parameter defined by an upper parameter boundary and a lower parameter boundary; (b) based on signals received from the sensor, measure a volume of fluid through the exit member during a first time window; (c) compare the measured volume to an expected volume; (d) based on the comparison, alter an operating parameter during a second time window following the first time window, the altered operating parameter being within the parameter range; and (e) repeat (b)-(d) during the continuous pumping cycle. In some embodiments, (b) and (c) are repeated during (d).

The operating parameter(s) may be at least one of pressure, flow volume, flow rate, flow time, voltage, current, integrations of any of the foregoing, or differentiations of any of the foregoing. In particular, the control circuitry may be configured to implement a closed-loop controller for maintaining the operating parameter at a constant value during at least the first time window and at an altered constant value during the second time window. The control circuitry may be configured to also (a) compare a cumulative volume of fluid through the exit member over a plurality of time windows to an expected cumulative volume, and (b) alter the operating parameter during the second time window based on the first time window volume comparison and the cumulative volume comparison.

In some embodiments, the control circuitry is configured to alter the operating parameter only if a difference between the measured volume and the expected volume exceeds a stored threshold. The control circuitry may be configured to deliver multiple discrete boluses during a time window, or to vary the number of single boluses delivered during a time window.

In another aspect, the invention relates to a method of controlling an actual delivery volume of fluid to conform to a target delivery volume in a drug pump device comprising a drug reservoir, an exit member for fluidically connecting the reservoir with a drug injection site, and an electrolysis pump operable to continuously force fluid from the drug reservoir into the exit member during a continuous pumping cycle. In various embodiments, the method comprises (a) providing a range of an operating parameter, where the range is defined by an upper parameter boundary and a lower parameter boundary; (b) measuring a volume of fluid through the exit member during a first time window; (c) comparing the measured volume to an expected volume; (d) based on the comparison, altering an operating parameter during a second time window following the first time window, the altered operating parameter being within the parameter range; and (e) repeating steps (b)-(d) during the continuous pumping cycle. In some embodiments, steps (b) and (c) are repeated during step (d).

The operating parameter(s) may be at least one of pressure, flow volume, flow rate, flow time, voltage, current, integrations of any of the foregoing, or differentiations of any of the foregoing. In particular, the operating parameter may be maintained at a constant value by closed-loop control during at least the first time window and at an altered constant value during the second time window; for example, the closed-loop control may be proportional-integral-derivative control.

In some embodiments, the method further comprises the steps of (a) comparing a cumulative volume of fluid through the exit member over a plurality of time windows to an expected cumulative volume, and (b) altering the operating parameter during the second time window based on the first time window volume comparison and the cumulative volume comparison. The operating parameter may, in some cases, be altered only if the difference between the measured volume and the expected volume exceeds a predefined threshold.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and can exist in various combinations and permutations. As used herein, the term “substantially” means ±10% and, in some embodiments, ±5%. A “measure” or “measurement” may be direct or indirect, i.e., a value derived from a directly measured value.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 is a block diagram illustrating the various functional components of electrolytic drug pump devices in accordance with various embodiments;

FIGS. 2A and 2B are schematic side views of piston pump devices in accordance with various embodiments;

FIG. 3 illustrates how repeated, alternating pulses and non-delivery periods can be combined to obtain an averaged flow rate equal to a targeted flow;

FIGS. 4A-4E graphically depict operation of an adaptive control algorithm for overcoming a flow “tail”;

FIG. 5, like FIG. 3, illustrates an example of discrete basal delivery achieved by multiple pulse/time deliveries;

FIG. 6 graphically depicts computation and use of average tail volumes in a compensating flow scheme at the priming stage;

FIG. 7 graphically depicts computation and use of average tail volumes in a compensating flow scheme at the delivery stage;

FIG. 8 graphically depicts the operation of an adaptive control algorithm for continuous basal delivery;

FIG. 9 graphically depicts the operation of an adaptive control algorithm for bolus delivery at the priming stage; and

FIG. 10A graphically depicts the delivery stage of the bolus algorithm; and

FIG. 10B graphically depicts the delivery stage of the bolus algorithm in a situation where the bolus volume is small and the peak flow rate never reaches the maximum dosing rate.

### DETAILED DESCRIPTION

### 1. Pump Architecture

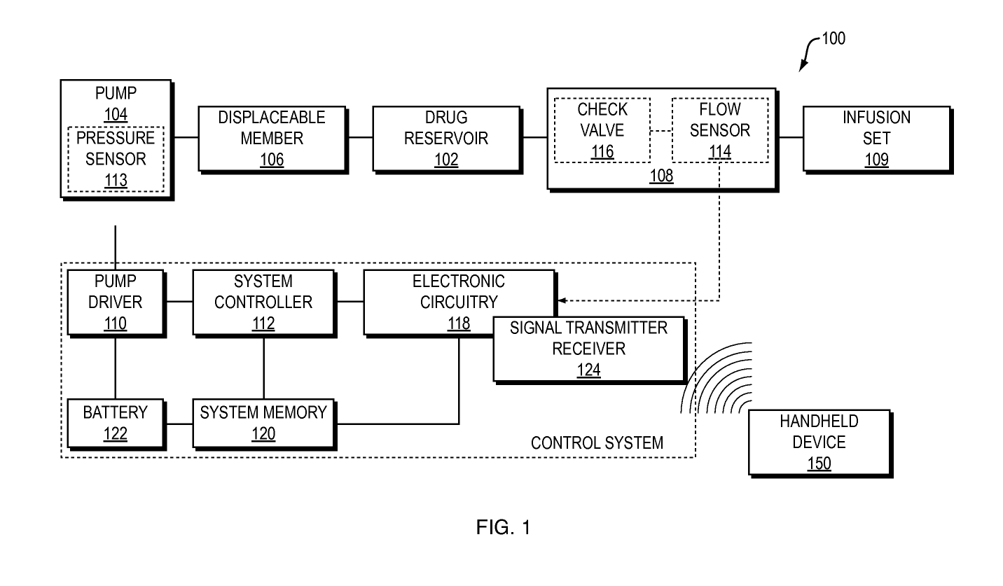

FIG. 1 illustrates, in block diagram form, the main functional components of a drug pump device 100 in accordance with various embodiments of the present invention. In general, the pump device 100 includes a drug reservoir 102 that interfaces with an electrolysis pump 104 via a displaceable member 106. The displaceable member 106 may be, for example, a piston, diaphragm, bladder, or plunger. In use, the drug reservoir 102 is filled with medication in liquid form, and pressure generated by the pump 104 moves or expands the displaceable member 106 so as to push the liquid drug out of the reservoir 102. A cannula, needle, or other exit member 108 connected to an outlet of the drug reservoir 102 conducts the liquid to an infusion set 109. The infusion set 109 may include a catheter fluidically connected to the cannula 108 for delivering the drug to a subcutaneous tissue region. A lancet and associated insertion mechanism may be used to drive the catheter through the skin. Alternatively, the infusion set 109 may include another type of drug-delivery vehicle, e.g., a sponge or other means facilitating drug absorption through the skin surface.

The electrolysis pump 104 generally includes an electrolyte-containing chamber (hereinafter also referred to as the “pump chamber”) and, disposed in the chamber, one or more pairs of electrodes that are driven by a direct-current power source to break the electrolyte into gaseous products. Suitable electrolytes include water and aqueous solutions of salts, acids, or alkali, as well as non-aqueous ionic solutions. The electrolysis of water is summarized in the following chemical reactions:

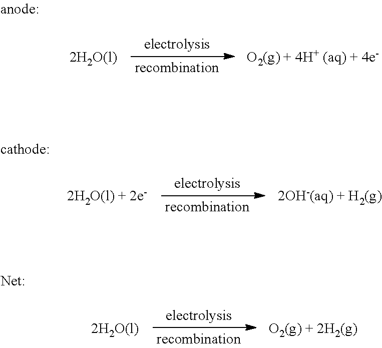

The net result of these reactions is the production of oxygen and hydrogen gas, which causes an overall volume expansion of the drug chamber contents. Gas evolution occurs even in a pressurized environment (reportedly at pressures of up to 200 MPa, corresponding to about 30,000 psi). As an alternative (or in addition) to water, ethanol may be used as an electrolyte, resulting in the evolution of carbon dioxide and hydrogen gas. Ethanol electrolysis is advantageous due to its greater efficiency and, consequently, lower power consumption, compared with water electrolysis. Electrolysis pumps in accordance with several embodiments are described in detail further below.

The pressure generated by the drug pump 104 may be regulated via a pump driver 110 by a system controller 112 (e.g., a microcontroller). The controller 112 may set the drive current and thereby control the rate of electrolysis, which, in turn, determines the pressure. In particular, the amount of gas generated is proportional to the drive current integrated over time, and can be calculated using Faraday's law of electrolysis. For example, creating two hydrogen and one oxygen molecule from water requires four electrons; thus, the amount (measured in moles) of gas generated by electrolysis of water equals the total electrical charge (i.e., current times time), multiplied by a factor of ¾ (because three molecules are generated per four electrons), divided by Faraday's constant.

The system controller 112 may execute a drug-delivery protocol programmed into the drug pump device 100, and may be responsive to one or more sensors 113, 114 that measure an operational parameter of the device 100, such as the pressure in the pump chamber 104 or the flow rate through (or pressure in) the cannula 108. For example, the controller 112 may adjust the current supplied to the electrolysis electrodes based on the pressure inside the pump chamber to achieve a target pressure. The target pressure, in turn, may be calculated based on a desired flow rate, using a known relationship between flow rate and pressure (as determined, e.g., by calibration). Due to the low cost of pressure sensors (such as, e.g., MEMS sensors as used in the automotive industry), this option is particularly advantageous for pumps designed for quick drug delivery. Indeed, two or more pressure sensors 113 may be placed in the pump chamber to simultaneously monitor pressure therein; this redundancy provides additional feedback to the controller 112, improves accuracy of information, and serves as a backup in case of malfunction of one of the sensors. Alternatively, the rate of drug flow out of the reservoir 102 may be measured directly and in real-time, using a flow sensor 114 integrated in the exit member 108 in a conventional manner. The total delivered dose can be computed by integrating the flow rate over time, and may serve as a control parameter for the electrolysis current as described in greater detail below.

In some embodiments, a pressure sensor 113 inside the pump chamber is used in combination with a flow sensor 114 in the cannula to increase the accuracy and precision of the feedback control loop. The use of multiple sensors also ensures that, in case the flow sensor 114 fails, the pressure sensor 113 would be able to detect high drug delivery rates, and shut the pump 104 down to avoid administration of an overdose to the patient or damage to the pump device. Conversely, the combination of flow and pressure sensors 114,113 can also detect a violation in the drug reservoir 102 if pressure is measured in the pump chamber but no flow is measured in the cannula 108, indicating a potential leak. In general, the sensors used to measure various pump parameters may be flow, thermal, time of flight, pressure, or other sensors known in the art, and may be fabricated (at least in part) from parylene—a biocompatible, thin-film polymer. The cannula 108 may also include a check valve 116 that prevents accidental drug delivery and backflow of liquid into the drug reservoir 112; like the sensor 114, the check valve 116 may be made of parylene. In other embodiments, silicon or glass are used in part for the flow sensor 114 and valve 116 construction.

The drug pump device 100 may include electronic circuitry 118 (which may, but need not, be integrated with the system controller 112) for conditioning and further processing the sensor signal(s) and, optionally, providing pump status information to a user by means of LEDs, other visual displays, vibrational signals, or audio signals. In addition to controlling the drug pump 104, the controller 112 may be used to control other components of the drug pump system; for example, it may trigger insertion of the lancet and catheter. The system controller 112 may be a microcontroller, i.e., an integrated circuit including a processor core, memory (e.g., in the form of flash memory, read-only memory (ROM), and/or random-access memory (RAM)), and input/output ports. The memory may store firmware that directs operation of the drug pump device. In addition, the device may include read-write system memory 120. In certain alternative embodiments, the system controller 112 is a general-purpose microprocessor that communicates with the system memory 120. The system memory 120 (or memory that is part of a microcontroller) may store a drug-delivery protocol in the form of instructions executable by the controller 112, which may be loaded into the memory at the time of manufacturing, or at a later time by data transfer from a hard drive, flash drive, or other storage device, e.g., via a USB, Ethernet, or firewire port. In alternative embodiments, the system controller 112 comprises analog circuitry designed to perform the intended function.

The pump driver 110, system controller 112, and electronic circuitry 118 may be powered, via suitable battery electronics, by a battery 122. Suitable batteries 122 include non-rechargeable lithium batteries approximating the size of batteries used in wristwatches, as well as rechargeable Li-ion, lithium polymer, thin-film (e.g., Li—PON), nickel-metal-hydride, and nickel cadmium batteries. Other devices for powering the drug pump device 100, such as a capacitor, solar cell or motion-generated energy systems, may be used either in place of the battery 122 or supplementing a smaller battery. This can be useful in cases where the patient needs to keep the drug-delivery device 100 on for several days or more.

In certain embodiments, the drug pump device 100 includes, as part of the electronic circuitry 118 or as a separate component, a signal receiver 124 (for uni-directional telemetry) or a transmitter/receiver 124 (for bi-directional telemetry) that allows the device to be controlled and/or re-programmed remotely by a wireless handheld device 150, such as a customized remote control or a smartphone. In certain embodiments, the handheld device 150 and pump device 100 communicate over a (uni- or bidirectional) infrared (IR) link, which may utilize one or more inexpensive IR light-emitting diodes and phototransistors as transmitters and receivers, respectively. Communication between the drug pump device 100 and the handheld device 150 may also occur at radio frequencies (RF), using, e.g., a copper coil antenna as the transmitter/receiver component 124.

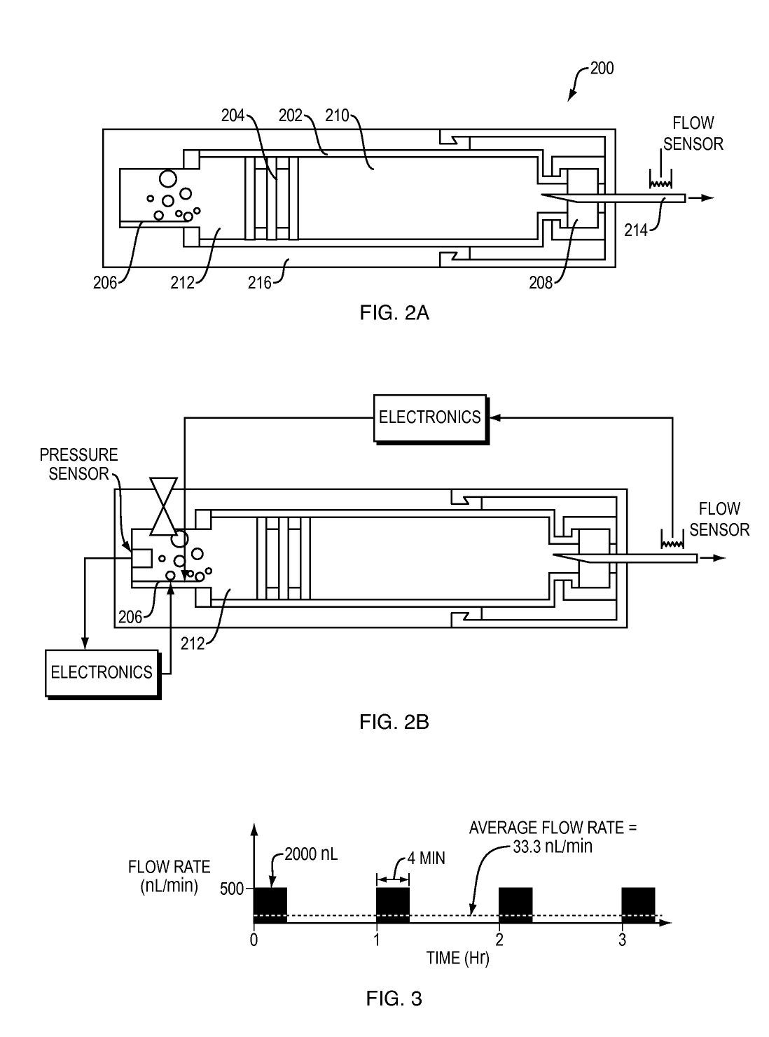

The drug-delivery device 100 may be manually activated, e.g., toggled on and off, by means of a switch integrated into the pump housing. In some embodiments, using the toggle switch or another mechanical release mechanism, the patient may cause a needle to pierce the enclosure of the drug reservoir 102 (e.g., the septum of a drug vial, as explained below with respect to FIGS. 2A and 2B) to establish a fluidic connection between the reservoir 102 and the cannula 108; priming of the pump can then begin. During priming, liquid is pumped from the reservoir through the fluid path, ideally displacing air with liquid up to the tip of the injection needle. Coupling insertion of the needle into the reservoir 102 with the activation of the pump device ensures the integrity of the reservoir 102, and thus protects the drug, up to the time when the drug is injected; this is particularly important for pre-filled drug pump devices. Similarly, the lancet and catheter of the infusion set 109 may be inserted by manually releasing a mechanical insertion mechanism. In some embodiments, insertion of the lancet and catheter automatically triggers electronic activation of a pump, e.g., by closing an electronic circuit. Alternatively, the pump and/or insertion set may be activated remotely by wireless commands.

The functional components of drug pump devices as described above may be packaged and configured in various ways. In certain preferred embodiments, the drug pump device is integrated into a patch adherable to the patient's skin. Suitable adhesive patches are generally fabricated from a flexible material that conforms to the contours of the patient's body and attaches via an adhesive on the backside surface that contacts a patient's skin. The adhesive may be any material suitable and safe for application to and removal from human skin. Many versions of such adhesives are known in the art, although utilizing an adhesive with gel-like properties may afford a patient particularly advantageous comfort and flexibility. The adhesive may be covered with a removable layer to preclude premature adhesion prior to the intended application. As with commonly available bandages, the removable layer preferably does not reduce the adhesion properties of the adhesive when removed. In some embodiments, the drug pump device is of a shape and size suitable for implantation.

The various components of the drug pump device may be held within a housing mounted on the skin patch. The device may either be fully self-contained, or, if implemented as discrete, intercommunicating modules, reside within a spatial envelope that is wholly within (i.e., which does not extend beyond in any direction) the perimeter of the patch. The housing may provide mechanical integrity and protection of the components of the drug pump device 100, and prevent disruption of the pump's operation from changes in the external environment (such as pressure changes). The control system components 110, 112, 118, 120, 122 may be mounted on a circuit board, which may be flexible and/or may be an integral part of the pump housing. In some embodiments, the control system components are integrated with the electrolysis electrodes into self-contained unit.

Drug pump devices 100 in accordance herewith may be designed for single or repeated use. Multi-use pumps generally include a one-way check valve and a flow sensor, as described above, in the cannula. Further, the drug reservoir of a multi-use pump may be refillable via a refill port, using, e.g., a standard syringe. In some embodiments, the drug pump device 100 is removed from the patient's skin for re-filling. The patient may, for example, place the drug pump device 100 and cartridge containing the new drug into a home refill system, where the pump device and cartridge may be aligned using, e.g., a press-machine mechanism. The patient may then press a button to trigger automatic insertion of a needle that draws liquid drug from the cartridge to the cannula in order to activate the electronics and begin priming the pump.

The electrolysis pump 104 and drug reservoir 102 may be arranged within the device 100 in different ways, the two most common being a piston-pump configuration, in which the pump chamber and reservoir are formed within an elongated vial and separated by a piston movable along the axis of the vial, and the diaphragm-pump configuration, in which the reservoir is disposed on top of the pump chamber and separated therefrom by a flexible diaphragm. Both configurations are described in detail in U.S. patent application Ser. No. 13/091,047, filed on Apr. 20, 2011, which is hereby incorporated herein by reference in its entirety.

FIG. 2A schematically illustrates an exemplary piston pump device 200. The pump device 200 includes a cylindrical (or, more generally, tubular) vial 202 with a piston 204 movably positioned therein and an electrolysis electrode structure 206 mounted to one end. A septum 208 may be disposed at the other end to seal the vial 202. Both the piston 204 and the septum 208 may be made of an elastomeric polymer material, such as a synthetic or natural rubber; in some embodiments, silicone rubber (i.e., polydiorganosiloxane, e.g., polydimethylsiloxane) is used. The piston 204 separates the interior of the vial 202 into a drug reservoir 210 and a pump chamber 212. In use, a needle 214 pierces the septum 208 to allow fluid egress from the drug reservoir 210; a cannula (not shown) connected to the needle 214 may conduct the fluid to the infusion set (not shown). The piston pump device 200 is enclosed in a protective housing 216, e.g., made of a hard plastic.

The electrodes 206 may be made of any suitable metal, such as, for example, platinum, titanium, gold, or copper, and may form a pair of parallel wires or plates. Alternatively, to improve electrolysis efficiency, the electrodes can have non-traditional shapes. For example, they may be interdigitated, or individually wound up into a spiral configuration (and oriented so as to face each other) as illustrated in FIG. 2B. Further, as shown, the electrodes 206 may be embedded in a hydrophilic absorbent material 218 (e.g., a cotton ball) that ensures continuous contact with the electrolyte 220. This solves a problem frequently encountered with conventional electrolysis pumps, in which the electrodes are simply submerged in liquid electrolyte: as gaseous electrolysis products are generated, they push the piston towards the outlet end of the drug reservoir, thereby increasing the volume of the electrolysis chamber, which causes a decrease in the level of the electrolyte. Depending on the orientation of the device, one or both electrodes may, as a result, gradually emerge from the electrolyte and become surrounded by the gas, eventually forming an open circuit and, thus, causing the electrolysis reaction to cease. This problem can be avoided in various ways, one of which is to surround the electrodes with a hydrophilic absorbent material such as (but not limited to) a hydrogel, cotton ball, sponge, or super-absorbent polymer. The electrolyte stays inside the hydrophilic absorbent material, which efficiently expels the generated gas and keeps the electrodes replenished with electrolyte.

The vial 202 may be fabricated from a glass, polymer, or other materials that are inert with respect to the stability of the drug and, preferably, biocompatible. Polymer vials, e.g., made of polypropylene or parylene, may be suitable for certain drugs that degrade faster when in contact with glass, such as protein drugs. For many other drugs, glass is the preferred material. Glass is commonly used in commercially available and FDA-approved drug vials and containers from many different manufacturers. As a result, there are well-established and approved procedures for aseptically filling and storing drugs in glass containers, which may accelerate the approval process for drug pump devices that protect the drug in a glass container, and avoid the need to rebuild a costly aseptic filling manufacturing line. Using glass for the reservoir further allows the drug to be in contact with similar materials during shipping. Suitable glass materials for the vial may be selected based on the chemical resistance and stability as well as the shatterproof properties of the material. For example, to reduce the risk of container breakage, type-II or type-III soda-lime glasses or type-I borosilicate materials may be used.

To enhance chemical resistance and maintain the stability of enclosed drug preparations, the interior surface of the vial may have a specialized coating. Examples of such coatings include chemically bonded, invisible, ultrathin layers of silicon dioxide or medical-grade silicone emulsions. In addition to protecting the chemical integrity of the enclosed drugs, coatings such as silicone emulsions may provide for lower and more uniform friction between the piston and vial.

In certain embodiments, the piston pump device 200 is manufactured by fitting a conventional, commercially available glass or polymer drug vial, which may already be validated for aseptic filling, with the piston 204 and electrolysis pump components. A screw-in needle cassette may be placed over the septum 208, and a mechanical actuation mechanism may serve to screw the cassette into the vial 202 such that the cassette needle 214 punctures the septum 208 and establishes a connection with the cannula at the time the patient desires to use the pump. To accommodate the electrolysis pump, the vial 202 is, in some embodiments, longer than typical commercially available vials, but maintains all other properties such that validated filling methods and the parameters of existing aseptic filling lines need not be changed. The drug pump device may be furnished with a prefilled vial. If a glass vial is used, the drugs can be stored in the pump device for long-term shelf life without the need to change the labeling on the drug.

FIG. 2B illustrates the pump 200 with a pressure sensor located in the pump chamber 212. Signals from the flow sensor and the pressure sensor are received by programmable circuitry and may be used to regulate pump operation as described in detail below.

### 2. Feedback Control

Closed-loop feedback control in accordance herewith ensures accurate drug delivery in driven pumps. In one delivery scheme, a pump system delivers drug in discrete doses or pulses, resulting in flow rates that are much lower than the continuous delivery capabilities of the pump. Due to the effect of the stiction of the plunger and glass wall of the cartridge at such a low flow rate, each small discrete dose of drug is generated after the plunger is pushed to overcome the stiction force. Each discrete delivery may overshoot to a flow rate higher than a targeted flow rate, followed by an abrupt stop of the plunger movement causing the flow rate to cease for certain interval. Combining these repeated, alternating pulses and non-delivery periods results in an averaged flow rate theoretically equal to the targeted flow rate and the amount of dose volume equal to the volume obtained from continuous delivery mode. This approach is illustrated in FIG. 3, which shows a discrete dose mode in which the peak flow rate for each pulse is 500 mL/min, the duration for each pulse is 4 min, and the average flow rate representing a continuous constant delivery mode is 33.3 mL/min.

The mechanics of actually delivering these discrete doses, however, can result in inaccuracy (such as an overdose or underdose relative to the desired target delivery). When electrolysis occurs in an electrolytically driven plunger pump, pressure builds up behind the plunger, causing it to compress. When electrolysis stops, however, the plunger relaxes from this compressed state. The relaxation of the plunger material continues to push fluid out of the drug reservoir, causing a prolonged “tail” of the flow rate. This residual flow eventually ceases after the plunger returns to its natural state. The flow sensor enables the system to determine the actual dose delivered, triggering a control algorithm (such as an artificial neural network, fuzzy logic, etc.) that accounts for deviations from the target dose. In other embodiments, a pressure sensor in the pump chamber is used instead of, or in addition to, the flow sensor, since pressure readings are readily correlated with the volume of fluid expelled from the reservoir.

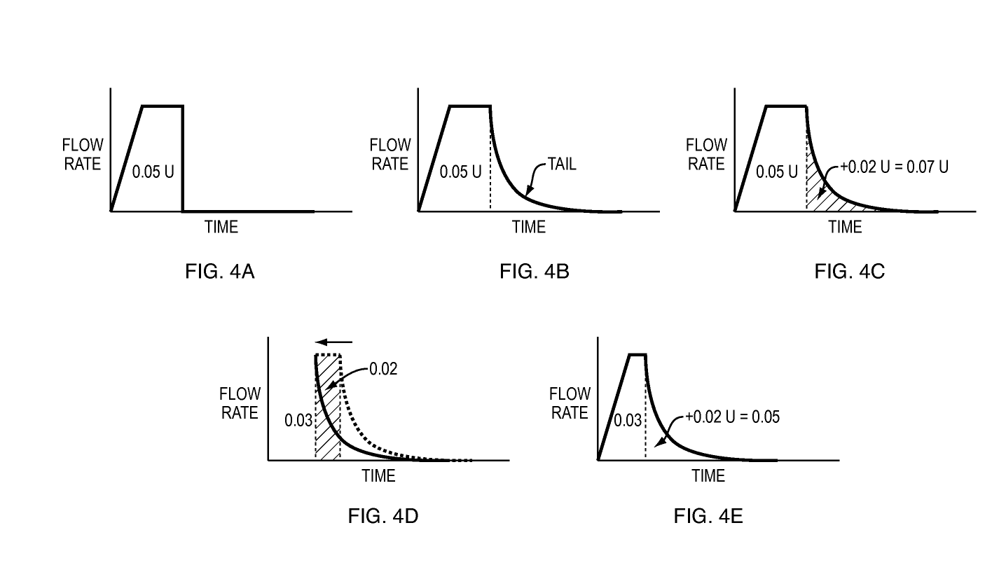

FIGS. 4A-4E illustrate the general concept of an adaptive control algorithm suitable for addressing this problem. In particular, FIG. 4A shows an ideal discrete dose of 0.05 U (which deviates from a perfectly rectangular pulse due to a necessary ramp-up time, which represents proper pump operation and does not vary significantly); FIG. 4B shows the actual delivered dose with additional volume “tail”; FIG. 4C illustrates measurement of the tail at 0.02 U, which totals 0.07 U delivered; the pump stops delivery early at 0.03 U, which accounts for the unwanted tail of 0.02 U shown in FIG. 4D (i.e., the pulse is adjusted so that the tail volume becomes part of the intended delivery volume rather than a deviation therefrom); and FIG. 4E illustrates performance in accordance with embodiments of the present invention, which results in a delivered dose that more accurately tracks the target dose shown in FIG. 4A.

A typical insulin pump should be able to provide bolus and background basal delivery rates over a wide range in order to serve different patients' needs. A prefilled insulin pump in accordance herewith can successfully provide suitable basal and bolus ranges. However, due to the nature of prefilled cartridge pumps, in particular the varying friction between the rubber plunger and glass wall among different cartridges, delivery accuracies can be compromised if corrections for such variation are not made. Thus, a real-time intelligent control algorithm may be used to compensate for the variation and maintain a very accurate dosage for both basal and bolus delivery.

Due to the unpredictable interaction between the plunger and glass cartridge of a driven pump, many variables can contribute to dosing inaccuracies. Since an initial stiction (initial static friction) exists between the plunger and glass vial, the pump must achieve a minimum pressure before the plunger can move smoothly to deliver a truly continuous flow rate. In this situation, only dynamic friction occurs between plunger and the glass container. On the other hand, the initial stiction (or static friction) may limit the minimum flow rate that the pump can offer and make it very difficult to deliver small amount of drug. To deliver at flow rates below this threshold, a discrete delivery scheme herein termed “discrete basal delivery” may be utilized. In this scheme, the target flow rate is converted to a target volume delivery in a given time period. Several small pulses of insulin are delivered throughout the given time span to achieve the target volume delivery, which fulfills the targeted average flow rate as described in the following equation.

$$Flow Rate ( \frac{U}{Hr} ) = \frac{Total Delivery Volume ( U )}{Time ( Hr )} \qquad ( 3 )$$

In one representative embodiment, the volume of the tail is measured from the previous few (e.g., three) doses the average is determined. The system controller 112 then adjusts the target volume for the next dose based on this average. The following equations may be used by the controller to determine the proper correction.

$$\begin{matrix}{V}_{Tail\_Avg} = \frac{{\sum }_{k = 0}^{n} {V}_{Tail} ( k )}{n} & ( 1 ) \\ {V}_{set} ( n ) = {V}_{target} ( n ) - {V}_{Tail\_Avg} & ( 2 )\end{matrix}$$

where V_{Tail}_{_{—}}_{Avg} is the average tail volume, V_{Tail} is the individual tail volume, Vₛₑₜ is the predicted volume to be delivered excluding the tail volume, and V_{target} is the total expected volume.

A similar approach may be used for continuous delivery. In particular, after analyzing the pump's previous average delivery during a given time period, the system controller 112 adjusts the overall flow rate (on a pulse-by-pulse basis) to correct for the previous delivery error during the next time period.

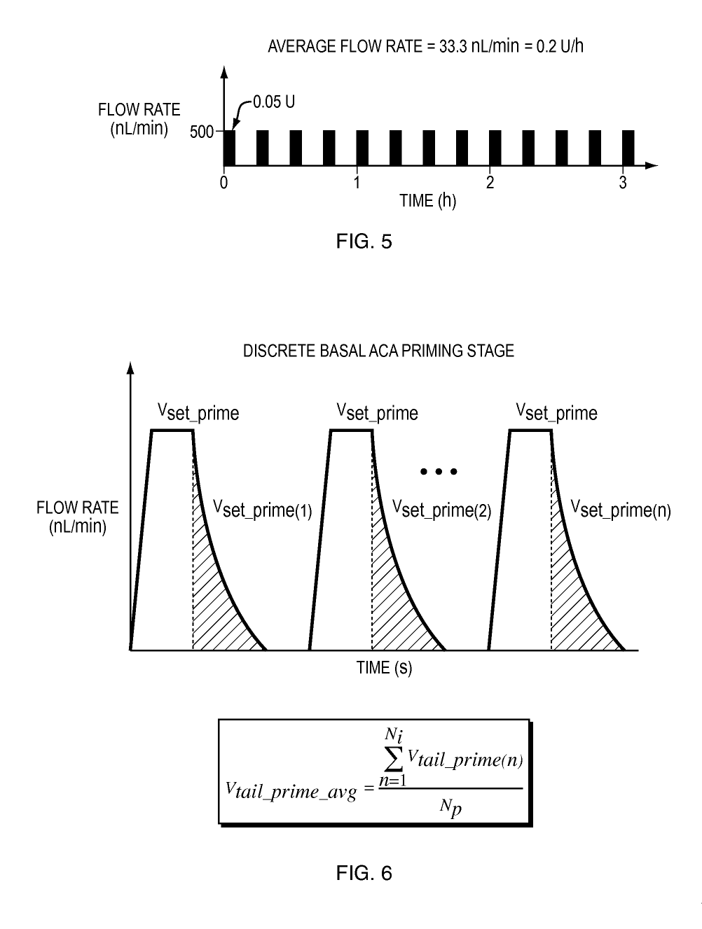

FIG. 5 illustrates an example of discrete basal delivery achieved by multiple pulse/time deliveries. As shown, there are four bolus deliveries in an hour and the equivalent flow rate is 33.3 mL/min. To generate these small pulses, the pressure in the electrolysis chamber is quickly released at the end of each pulse to accurately obtain the targeted volume for each small pulse. If the pressure is not promptly released, the high pressure will prevent the pump flow from stopping and result in a large over-delivery. Meanwhile, due to residual pressure induced from the plunger/glass interaction and the compliance of the plunger, a “tail” is produced that degrades the accuracy of basal delivery.

In one embodiment that corrects for these errors, the system controller 112 executes an adaptive control algorithm (ACA) during a priming stage and during a delivery stage. At the priming stage, a number n of predetermined pulses (e.g., 0.05 U/pulse) is scheduled every t minutes. After reaching the target volume for each pulse, the pressure is released and the tail volumes measured. Throughout the priming stage, the system controller 112 (see FIG. 1) collects these tail volumes from a number Nₚ of pulses to determine an average tail volume. This averaged volume is taken into account in adjusting the time width of the subsequent pulse during the delivery stage. The priming tails are averaged and stored as Vₜₐᵢₗ_{_{—}}ₚᵣᵢₘₑ_{_{—}}_{avg} for future usage. This is illustrated in FIG. 6, in which Nₚ is the number of discrete basal pulses to be delivered during the priming stage, Vₜₐᵢₗ_{_{—}}ₚᵣᵢₘₑ(n) corresponds to the n^{th} tail volume generated by the n^{th} pulse in the priming stage, and Vₜₐᵢₗ_{_{—}}ₚᵣᵢₘₑ_{_{—}}_{avg} corresponds to the average tail volume of the discrete pulses in the priming stage.

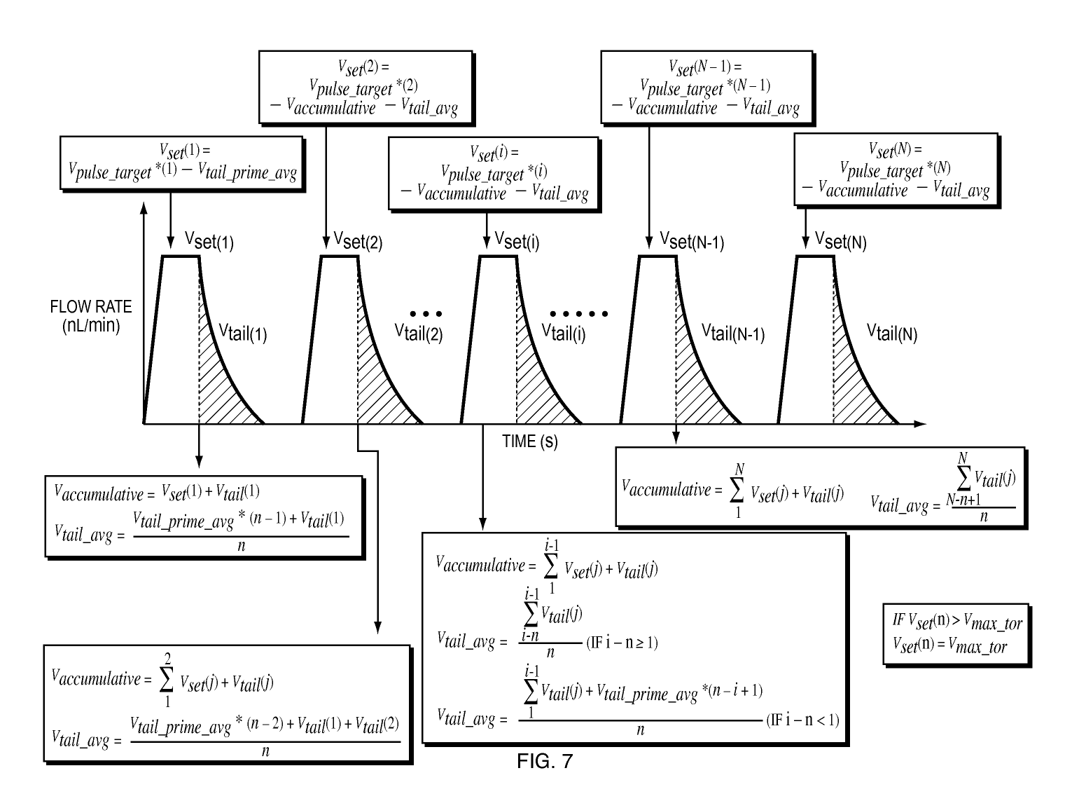

At the delivery stage, as illustrated in FIG. 7, the discrete basal target volume is adjusted according to the average volume of the previous n tails. The first discrete basal pulse, Set Volume or Vₛₑₜ(n), is established as follows. After the tail volumes from the n pulses during the priming stage are averaged, Vₜₐᵢₗ_{_{—}}ₚᵣᵢₘₑ_{_{—}}_{avg} is subtracted from V_{target} (the original target volume of the pulse) to give Vₛₑₜ(1) (i.e., the adjusted volume for the pulse). In FIG. 7, Vₚᵤₗₛₑ_{_{—}}_{target} is the target volume to be delivered in each pulse, Vₜₐᵢₗ(n) is the tail volume produced by each pulse, V_{cumulative} is the actual cumulative volume delivered over time, and Vₜₐᵢₗ_{_{—}}_{avg} is the average tail of the previous n discrete basal pulses.

For each subsequent discrete basal pulse, the system uses the previous n tail volumes in calculating the average tail volume to be used in determining the Vₛₑₜ for the current discrete basal pulse. For example, if there are three discrete pulses delivered during the priming stage (n=3), Vₜₐᵢₗ_{_{—}}_{avg} for the first pulse in the delivery stage corresponds to the average of these three previous tails, which in this case is the average of these three tail volumes during the priming stage. Next, the value of Vₜₐᵢₗ_{_{—}}_{avg} for the second pulse in the delivery stage corresponds to the average of the three previous tails. However, the three previous tails are the second and third discrete basal pulses in the priming stage and the first pulse of the delivery stage. Vₜₐᵢₗ_{_{—}}_{avg} for the third pulse in the delivery stage corresponds to the average of the third tail in the priming stage and the first and second tails of the delivery stage, and so forth.

A special case arises when Vₛₑₜ is negative. This means that there is an overdose from a previous delivery such that the next scheduled pulse should deliver a negative volume to achieve the desired target volume, V_{target}_{_{—}}ₒᵥₑᵣₐₗₗ. Of course, drug pumps ordinarily cannot operate to withdraw fluid from the patient. Instead, the system sets Vₛₑₜ to 0, which causes the controller 112 to skip the pulse in an effort to correct for a previous over-delivery and achieve the correct overall target volume, V_{target}_{_{—}}ₒᵥₑᵣₐₗₗ. Also, to reduce any over-delivery caused by missing multiple pulses, the system can set a maximum tolerance volume to be delivered at each pulse (Vₘₐₓ_{_{—}}ₜₒᵣ). The Vₛₑₜ volume never exceeds Vₘₐₓ_{_{—}}ₜₒᵣ. If it does, the system controller 112 will coerce Vₛₑₜ to be equal to Vₘₐₓ_{_{—}}ₜₒᵣ. This ensures overall profile stability, and also ensures the absence of dramatic change in overdose and underdose caused by frequent over-delivery and missed pulses.

It should be noted that the tail volume can vary over the lifetime of the pump or even during an operating cycle based on various factors. Accordingly, calibration is typically performed periodically rather than, for example, a single time when the pump is first used. For example, the response of an electrolysis-driven pump to a given input current supplied to the electrolysis electrodes depends on how much liquid is remaining in the drug reservoir and the gas/liquid ratio in the electrolysis chamber. Other factors can cause the response of the pump to change over time including, for example, degradation of electrolysis electrodes, changes in the concentration of the electrolyte in the electrolysis chamber, changes in the flow characteristics of valves in the fluid path, and restrictions that form at the exit port due to tissue growth or some other mechanism.

Similar to discrete basal delivery, continuous basal delivery faces challenges due to the plunger and glass-wall friction. Ordinarily it is not necessary to deliver a pulse pattern in continuous basal delivery (as contrasted with discrete basal delivery), and during continuous pumping, the plunger operates above its stiction range. Nonetheless, a continuous adaptive control algorithm can increase the accuracy of drug delivery by minimizing the errors caused by interaction between the plunger and the glass wall.

In one embodiment, a continuous adaptive control routine continuously monitors the accumulated volume and its deviation from the target volume. The routine sets a time window, ΔT, and maximum tolerable flow rate range, ΔQₘₐₓ_{_{—}}ₜₒᵣ. The routine calculates the actual volume delivered during ΔT and its deviation from the target volume. Based on the deviation, a target flow rate, Qₛₑₜ(2), for the next ΔT window is determined in order to compensate for the error in delivery during the first ΔT window. The maximum tolerance, ΔQₘₐₓ_{_{—}}ₜₒᵣ, comes into play when there is too much error in delivery during the first ΔT and Qₛₑₜ(2) has been raised or decreased beyond the physiological range from Qₛₑₜ_{_{—}}ᵢₙᵢₜᵢₐₗ. In such case, Qₛₑₜ(2) is coerced to equal Qₛₑₜ_{_{—}}ᵢₙᵢₜᵢₐₗ±ΔQₘₐₓ_{_{—}}ₜₒᵣ, depending on the delivery error during previous sampling time. This process repeats throughout continuous basal delivery to ensure the overall stability of the flow profile and delivery accuracy.

There are many ways to raise the Qₛₑₜ for the next ΔT time window. A simple example is to have only three possible values for Qₛₑₜ: Qₛₑₜ_{_{—}}ᵢₙᵢₜᵢₐₗ, Qₛₑₜ_{_{—}}ᵢₙᵢₜᵢₐₗ+ΔQₘₐₓ_{_{—}}ₜₒᵣ, and Qₛₑₜ_{_{—}}ₘᵢₙ−ΔQₘₐₓ_{_{—}}ₜₒᵣ. In one embodiment, if the cumulative delivered volume did not deviate from the cumulative target volume by more than an acceptable percentage (e.g., 5%) at the end of the sampling period ΔT, then the system will set Qₛₑₜ to Qₛₑₜ_{_{—}}ᵢₙᵢₜᵢₐₗ, which is the initial flow-rate set point. If the actual delivered volume exceeds the target volume by more than the acceptable percentage (e.g., 5%), Qₛₑₜ is set to Qₛₑₜ−ΔQₘₐₓ_{_{—}}ₜₒᵣ for the next time window ΔT. If the actual delivered volume falls below the target volume by more than the acceptable percentage (e.g., 5%), Qₛₑₜ is set to Qₛₑₜ+ΔQₘₐₓ_{_{—}}ₜₒᵣ for the next time window ΔT.

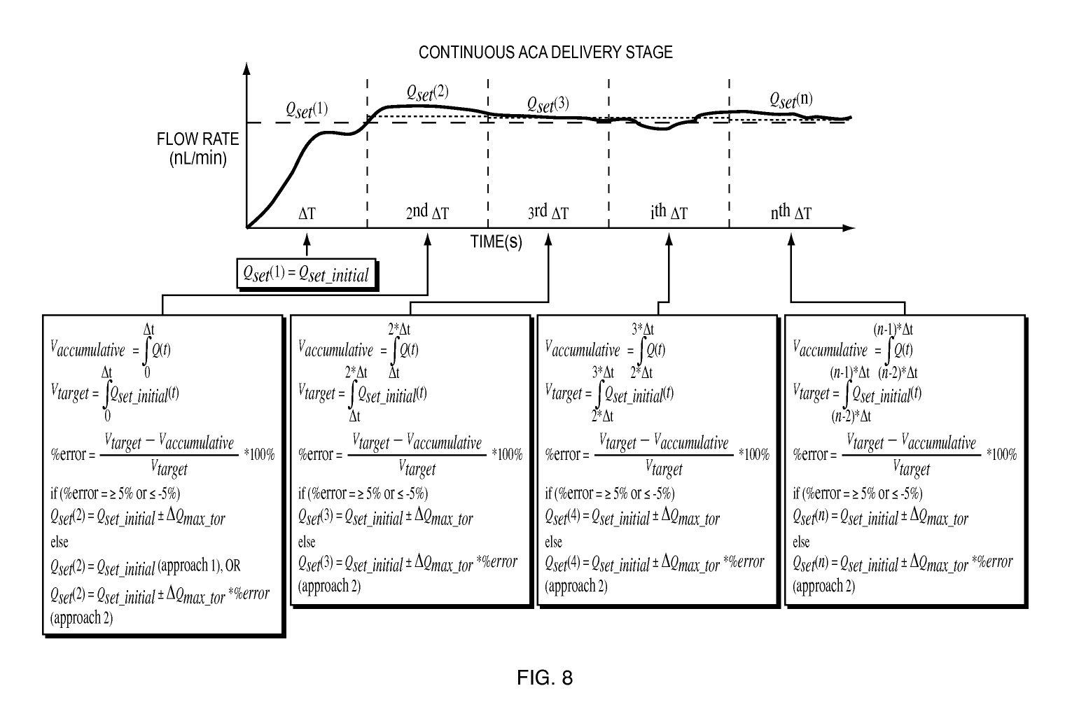

In another embodiment, illustrated in FIG. 8, a continuous spectrum of Qₛₑₜ(n) values is used. In the figure, ΔT is a predefined time window, Qₛₑₜ(n) is the target flow rate during the time window, Qₛₑₜ_{_{—}}ᵢₙᵢₜᵢₐₗ is the initial target flow rate (which is also equal to Qₛₑₜ, the overall target flow rate), V_{cumulative} is the actual cumulative volume delivered over time, and V_{target} is the target cumulative volume over time, % error represents the error percentage (i.e., the deviation from V_{target}) by volume, and ΔQₘₐₓ_{_{—}}ₜₒᵣ is the maximum flow rate that Qₛₑₜ cannot exceed.

Each Qₛₑₜ(n) value inversely corresponds to a percentage deviation above or below the target delivery volume from the previous sampling period. If the cumulative delivered volume exceeds the cumulative target volume by more than an acceptable percentage (% error), Qₛₑₜ(n) is set to Qₛₑₜ_{_{—}}ᵢₙᵢₜᵢₐₗ−% error×ΔQₘₐₓ_{_{—}}ₜₒᵣ for the next time window ΔT. If the actual delivered volume falls below the target volume by more than % error, Qₛₑₜ(n) is set to Qₛₑₜ_{_{—}}ᵢₙᵢₜᵢₐₗ+% error×ΔQₘₐₓ_{_{—}}ₜₒᵣ for the next time window ΔT. Once again, the Qₛₑₜ(n) can never go below Qₛₑₜ_{_{—}}ᵢₙᵢₜᵢₐₗ−ΔQₘₐₓ_{_{—}}ₜₒᵣ or above Qₛₑₜ_{_{—}}ᵢₙᵢₜᵢₐₗ+ΔQₘₐₓ_{_{—}}ₜₒᵣ, or the system will coerce the Qₛₑₜ(n) to be set at Qₛₑₜ_{_{—}}ᵢₙᵢₜᵢₐₗ−ΔQₘₐₓ_{_{—}}ₜₒᵣ or Qₛₑₜ_{_{—}}ᵢₙᵢₜᵢₐₗ+ΔQₘₐₓ_{_{—}}ₜₒᵣ.

More generally, various algorithms and controllers can be used to adjust pump operation through monitoring and adjustment during time windows. During a time window, for example, a closed-loop control scheme, such as proportional-integral-derivative (“PID”) controller, on-off controller, fuzzy logic controller, proportional controller, and/or linear controller can be applied to maintain as constant a target delivery parameter such as flow rate. In the next time window, the constant target delivery parameter can be altered based on the comparison result from the previous time window For example, a PID controller may be used during a timing interval, while a different algorithm, such as the ACA, may be used between time windows to alter the PID controller settings (e.g., the constant target delivery flow rate, the acceptable upper and lower ranges of flow rates, and constants for positional, integral, and derivative calculations). Accordingly, parameters that can be monitored and adjusted include flow rate, pressure, volume, current, and voltage. Moreover, the integration or differentiation of any one or more of these parameters may be monitored and adjusted.

Bolus delivery faces the same accuracy challenges caused by the tail volume as the discrete basal pulses. However, boluses are delivered on demand, and the accuracy of each individual bolus cannot be compensated by another bolus. In the case of insulin delivery, boluses are usually preceded and followed by a background basal delivery; for example, a bolus may be administered just before mealtime, after which insulin is delivered at the background basal rate until the next bolus. In such applications the bolus adaptive control algorithm can be relatively simple, as it may be based solely on tail volume to achieve overall accuracy. In other scenarios, however, the flow profile may be more complex and/or unpredictable; for example, the rate of drug administration may be varied periodically or continuously based on the monitored value of a physiologic, environmental or blood-borne chemical concentration parameter.

In one embodiment, the real-time bolus adaptive control algorithm involves a priming stage and a delivery stage. During the priming stage, a bolus (e.g., 1 U) is delivered and the tail volume is measured until the flow rate reaches zero. This bolus is sufficient in volume to allow the system to pump the flow rate up to its maximum dosing rate (e.g., 30 U/Hr for insulin pump). If the user selects a smaller bolus volume, the peak bolus flow rate may never reach the maximum dosing rate (e.g., 30 U/Hr). If the user selects a large bolus volume, the peak bolus target flow rate can be kept at the maximum dosing rate (e.g., 30 U/Hr) using a flow-sensor-based closed-loop control system as described, for example, in copending application Ser. No. 13/680,828, filed on Nov. 19, 2012 (the entire disclosure of which is hereby incorporated by reference); it simply takes longer to finish the bolus.

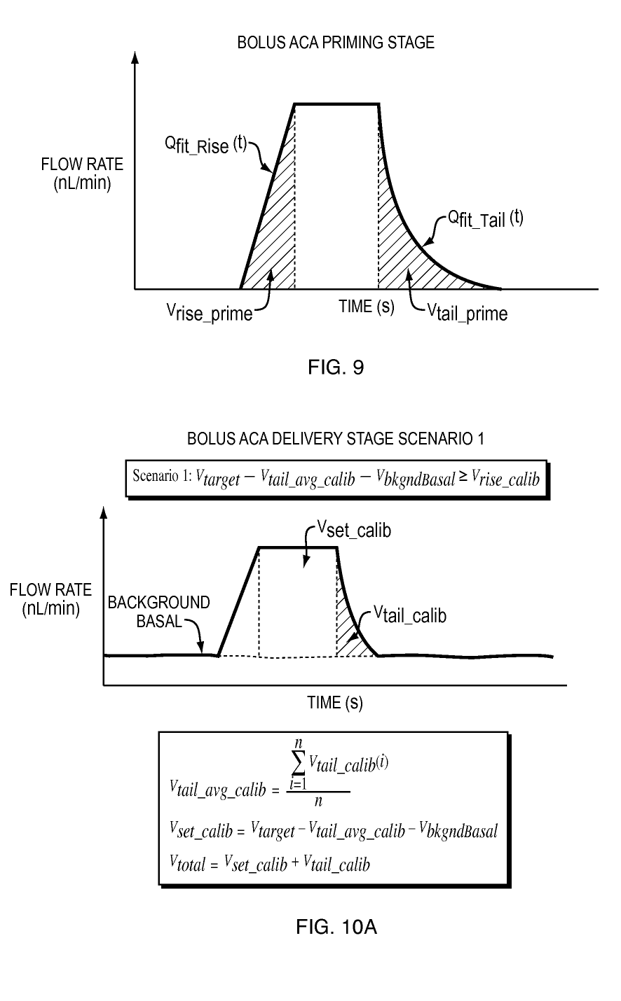

Operation during the priming stage is illustrated in FIG. 9. The white rectangle represents Vₛₑₜ_{_{—}}ₚᵣᵢₘₑ, i.e., the desired volume to be delivered. Additional flow during ramp-up (Vᵣᵢₛₑ_{_{—}}ₚᵣᵢₘₑ) and during the tail (Vₜₐᵢₗ_{_{—}}ₚᵣᵢₘₑ) is modeled by fitted curves Q_{fit}_{_{—}}_{Rise}(t) (flow rate vs. time during the ramp-up stage) and Q_{fit}_{_{—}}_{Rise}(t) (representing the tail after Vₛₑₜ_{_{—}}ₚᵣᵢₘₑ has completed). The areas under these fitted curves correspond to Vᵣᵢₛₑ_{_{—}}ₚᵣᵢₘₑ (the volume delivered during ramp-up) and Vₜₐᵢₗ_{_{—}}ₚᵣᵢₘₑ (the volume delivered during the tail segment). After obtaining the tail volume information and its curve, interpolation can be used to predict the tail volume for different boluses with different potential peak flow rates. The interpolated curve and the peak flow rate at the end of the bolus can be used to predict the tail volume.

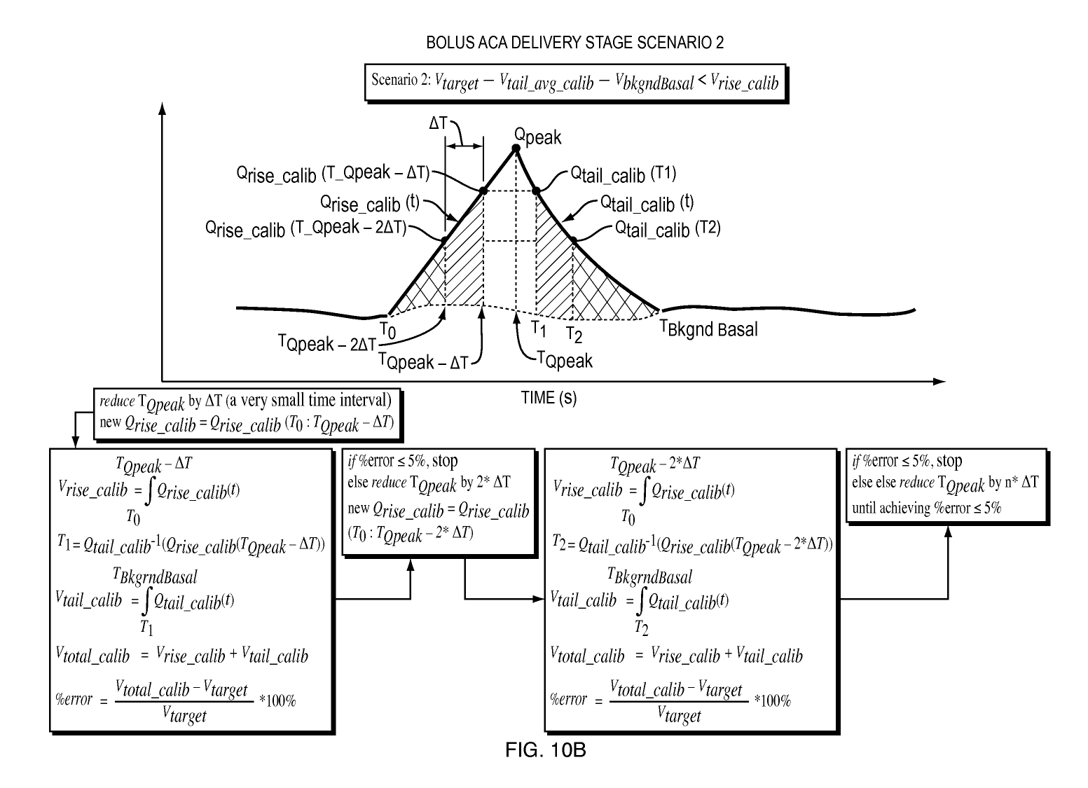

Two delivery-stage scenarios are illustrated, respectively, in FIGS. 10A and 10B. In FIG. 10A, the flow pattern corresponds to that shown in FIG. 9, with a peak flow rate is that is substantially constant (flat). A calibrated tail volume Vₜₐᵢₗ_{_{—}}_{avg}_{_{—}}_{calib} is used to calculate the actual delivery bolus volume, which is equal to the difference between the target volume V_{target} and the total predicted tail volume (including the background basal cumulative volume); thus, the calibrated set volume Vₛₑₜ_{_{—}}_{calib} is equal to V_{target}−Vₜₐᵢₗ_{_{—}}_{avg}_{_{—}}_{calib}−V_{BkgndBasal}. The total bolus volume Vₜₒₜₐₗ actually delivered equal to Vₛₑₜ_{_{—}}_{calib}+Vₜₒₜₐₗ_{_{—}}_{calib}, where Vₜₐᵢₗ_{_{—}}_{calib} is the actual run-time tail volume (this time ignoring the background basal cumulative volume).

If the bolus volume is small and the peak flow rate never reaches the maximum dosing rate (e.g., 30 U/Hr) before the target bolus volume has been delivered, a curve-fitting technique may be used to predict the peak flow rate as illustrated in FIG. 10B; here the tail volume is estimated between the predicted peak flow rate and the (actual) background basal flow rate. This can give a very accurate prediction of the tail volume, and during the actual delivery this is used to compensate for the effect of the tail effect. In FIG. 10B, T₀ is the bolus start time; T_{Qpeak} is the time at which the bolus reaches the maximum peak set flow rate Qₚₑₐₖ; ΔT is an adjustment time set to create a fitted curve as shown in FIG. 10B (and may range from, e.g., 1 ms to 1 s depending on the application in order to balance acceptable error and power restrictions that determine the sample rate and, in turn, the duration of the sample time interval); T₁ is the time when Qₜₐᵢₗ_{_{—}}_{calib}(t) (the fitted tail curve) reaches Qₜₐᵢₗ_{_{—}}_{calib}×(T_{Qpeak}−ΔT); T₂ is the time when Qₜₐᵢₗ_{_{—}}_{calib}(t) reaches Qₜₐᵢₗ_{_{—}}_{calib}×(T_{Qpeak}−2ΔT); and T_{BkgndBasal} is the time when Qₜₐᵢₗ_{_{—}}_{calib}(t) reaches the background basal flow rate.

After delivery is complete, the tail volume is stored in memory along with the estimated (fitted) curve. This historical tail behavior may be used to predict the tail volume for future boluses delivered from the same cartridge.

As discussed above, the priming stage is typically employed to evacuate air from the fluid path of the device, preventing air and/or debris from being injected into the target site, and also wets any sensors in the fluid path. Because of the faults that priming is designed to remediate, calibrating during the priming stage may not be ideal. In some embodiments, therefore, a non-therapeutic dose is dispensed by the pump following priming, and this dose is used instead of or in addition to the priming stage for calibration purposes. For example, it may be necessary to use the non-therapeutic dose for calibration if the sensor used therefor is in the fluid path and must be wet to operate properly. As used herein, the term “non-therapeutic dose” means a volume of drug less than a therapeutic dose, and in some embodiments, a dose small enough to avoid any therapeutic effect or clinically significant effect.

It should be emphasized that, although the preceding discussion has focused on a single target dosage, this need not be the case. Many medications, including monoclonal antibodies, require dosages based on the patient's weight or the severity of the diseases. Accordingly, pumps in accordance herewith may have the ability to deliver a range of target dosages; in some embodiments, a dose-selection mechanism is incorporated—for example, a dose-selection interface may allow the user to select the dosage, which is programmed into memory within the controller 112. This interface may be or include a switch, dial, buttons, touch screen, or a variety of user-interface components. The dosage may also be pre-set by the manufacturer, clinician, pharmacist, or other non-patient entity, and locked for security purposes.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A drug pump device comprising:
a drug reservoir;
an exit member for fluidically connecting the reservoir with a drug injection site;
a flow sensor;
an electrolysis pump operable to continuously force fluid from the drug reservoir into the exit member during a continuous pumping cycle; and
control circuitry configured to:
(a) store a range of an operating parameter defined by an upper parameter boundary and a lower parameter boundary;
(b) based on signals received from the sensor, measure a volume of fluid through the exit member during a first time window;
(c) compare the measured volume to an expected volume;
(d) based on the comparison, alter an operating parameter during a second time window following the first time window, the altered operating parameter being within the parameter range; and
(e) repeat (b)-(d) during the continuous pumping cycle.

2. The device of claim 1, wherein (b) and (c) are repeated during (d).

3. The device of claim 1, wherein the one or more operating parameters are at least one of pressure, flow volume, flow rate, flow time, voltage, current, integrations of any of the foregoing, or differentiations of any of the foregoing.

4. The device of claim 1, wherein the control circuitry is configured to implement a closed-loop controller for maintaining the operating parameter at a constant value during at least the first time window and at an altered constant value during the second time window.

5. The device of claim 1, wherein the control circuitry is configured to also (a) compare a cumulative volume of fluid through the exit member over a plurality of time windows to an expected cumulative volume, and (b) alter the operating parameter during the second time window based on the first time window volume comparison and the cumulative volume comparison.

6. The device of claim 1, wherein the control circuitry is configured to alter the operating parameter only if a difference between the measured volume and the expected volume exceeds a stored threshold.

7. The device of claim 1, wherein the control circuitry is configured to deliver multiple discrete boluses during a time window.

8. The device of claim 7, wherein the control circuitry is configured to vary a number of single boluses delivered during a time window.

9. In a drug pump device comprising a drug reservoir, an exit member for fluidically connecting the reservoir with a drug injection site, and an electrolysis pump operable to continuously force fluid from the drug reservoir into the exit member during a continuous pumping cycle, a method of controlling an actual delivery volume of fluid to conform to a target delivery volume, the method comprising:
(a) providing a range of an operating parameter, the range being defined by an upper parameter boundary and a lower parameter boundary;
(b) measuring a volume of fluid through the exit member during a first time window;
(c) comparing the measured volume to an expected volume;
(d) based on the comparison, altering an operating parameter during a second time window following the first time window, the altered operating parameter being within the parameter range; and
(e) repeating steps (b)-(d) during the continuous pumping cycle.

10. The method of claim 9, wherein steps (b) and (c) are repeated during step (d).

11. The method of claim 9, wherein the one or more operating parameters are at least one of pressure, flow volume, flow rate, flow time, voltage, current, integrations of any of the foregoing, or differentiations of any of the foregoing.

12. The method of claim 9, wherein the operating parameter is maintained at a constant value by closed-loop control during at least the first time window and at an altered constant value during the second time window.

13. The method of claim 12, wherein the closed-loop control is proportional-integral-derivative control.

14. The method of claim 9, further comprising the steps of (a) comparing a cumulative volume of fluid through the exit member over a plurality of time windows to an expected cumulative volume, and (b) altering the operating parameter during the second time window based on the first time window volume comparison and the cumulative volume comparison.

15. The method of claim 9, wherein the operating parameter is altered only if a difference between the measured volume and the expected volume exceeds a predefined threshold.
